(19) United States
(12) Patent Application Publication
(10) Pub. No.: US 2003/0163161 A1
(43) Pub. Date: Aug. 28, 2003

(54) Bone anchor protective cover

(76) Inventors: John Christopher Barron, Boston, MA (US); Ghaleb A. Sater, Lynnfield, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/804,498

(22) Filed: Mar. 12, 2001

(51) Int. Cl. A61B 17/04

(52) U.S. Cl. 606/232

(57) ABSTRACT

Low profile, bioabsorbable, bone anchor protective covers are disclosed for isolating bone anchors to reduce accidents with the sharp bone anchor tip and/or edges before it is inserted into a target site. Protective covers with antimicrobial material to reduce contamination of the target site by insertion of the bone anchor therethrough are also disclosed herein. The protective covers of the present invention are adapted to reduce accidents, for example, puncture of a doctor's glove, abrasion of the patient's tissue or insertion of the bone anchor tip into tissue other than the target tissue.

### FIELD OF THE INTENTION

This invention relates to surgical instruments in general and, more particularly, to protective covers for a bone anchor.

### BACKGROUND OF THE INVENTION

Bone anchors for attaching sutures, tissue, and/or bone to bone in various medical, veterinary, and dental surgical procedures, are known in the art. Also known are implantation devices for inserting such bone anchors into bone. Such devices may include mechanical devices to protect the leading tip of a bone anchor, such as plastic, spring-biased retractable sheaths.

In one surgical procedure, bone anchors may be attached to the pubic bone in procedures to treat female urinary incontinence. In such a procedure, female urinary incontinence may be treated by introducing suture-carrying anchors through the vaginal cavity, anchoring them into the posterior portion of the pubic bone or symphysis pubic, and attaching the anchored suture directly to the endopelvic fascia, the vaginal wall, or alternatively, to a sling, or other material to stabilize and/or slightly compress the urethra to thereby improve the patient's urinary continence.

In another surgical procedure using bone anchors, a small bone anchor may be placed in the bone behind the ear during an installation of a bone-anchored hearing aid, whereby the bone acts as a pathway for sound to travel to the inner ear without involving the ear canal.

In yet another surgical procedure using bone anchors, surgical realignment of the first metatarsal and great toe may be accomplished using a bone anchor to treat a bunion.

Surgical insertion of a bone anchor entails placing a small sharp object into a patient's bone, often in hard-to-reach sites. Such procedures are susceptible to accidental sticks or drops resulting in injuries to the patient and/or the surgeon. The devices known in the art for protecting a bone anchor are plastic cylindrical sheaths. Such cylindrical sheaths may significantly extend beyond the length and width of the bone anchor. Such a high profile configuration may unnecessarily abrade tissue during insertion of the bone anchor loaded in a bone anchor insertion device. Also known in the art are cylindrical sheaths used in conjunction with protective balloons placed over the bone anchor. In such devices, the bone anchor penetrates through such a protective balloon during the insertion into the bone. Such cylindrical sheaths and protective balloons are typically made from non-bioabsorbable materials. Such non-bioabsorbable sheaths and balloons present a danger to the patient if the cylindrical sheath or the protective balloon remain, in part or in whole, in the body. Presence of such polymers in the patient's body may, for example, increase the risk of an allergic reaction. Such danger can be especially severe if a particular patient is hypersensitive to certain types of non-bioabsorbable polymers, such as rubber or latex.

Moreover, during the implantation procedure it is desirable to prevent infection at the site where the bone anchor is introduced into the bone by providing supplemental antimicrobial protection of the bone anchor. This can be achieved most effectively if the bone anchor is covered with an antibiotic prior to, during, and/or after the implantation procedure. Cylindrical sheaths are typically hollow and not airtight. Antibiotics, which typically come in liquid or gelatinous form, may flow away from the bone anchor thereby leaving it exposed to the environment. As a result, before the surgical insertion, the bone anchor may come into contact with airborne bacteria, microorganisms present in the patient's body and/or other minute foreign particles, which present a risk of infection and biological complications if introduced into the bone during implantation of the bone anchor. Although cylindrical sheaths used in conjunction with protective balloons filled with an antibiotics may be more effective than cylindrical sheaths without antibiotics in isolating the bone anchor prior to the implantation procedure, such a sheath/balloon combination does not provide for antimicrobial protection after the bone anchor penetrates the balloon and is inserted into the bone. Accordingly, protective covers known in the art typically do not allow for supplemental antimicrobial protection of the bone anchor and the implantation site prior to, during, and after the implantation procedure.

Finally, the height of cylindrical sheaths known in the art may extend substantially beyond the length and width of the bone anchor. When the volume of such a protective sheath is substantially larger than the bone anchor protected by such sheath, the sheath is said to have a high profile in comparison to the bone anchor. Accordingly, using a high profile cylindrical sheath over the bone anchor complicates the implantation procedure by causing a larger wound or requiring a larger incision to access the target bone and also compromises ease of use by reducing maneuverability of the bone anchor implantation device. This is especially important when the bone anchor needs to be inserted into a bone through a narrowly constrained surgical field. Accordingly, there is a need in the art for a low profile, bioabsorbable, antimicrobial bone anchor protective cover.

### SUMMARY OF THE INVENTION

Low profile, bioabsorbable, antimicrobial bone anchor protective covers for isolating bone anchors to reduce accidents with the sharp bone anchor tip and/or edges before it is inserted into a target site, and to reduce contamination of the target site by insertion of the bone anchor therethrough, are disclosed herein.

The protective cover of the present invention is adapted to reduce accidents, for example, puncture of a doctor's glove, abrasion of the patient's tissue or insertion of the bone anchor tip into tissue other than the target tissue.

The protective cover of the present invention also provides a sterile antimicrobial barrier around the bone anchor and shields the bone anchor from contacting microorganisms in the surgical field surrounding the implantation site prior to and during insertion. The cover also provides prolonged supplemental antimicrobial protection of the implantation site after the bone anchor is inserted. Thus, the protective cover of the present invention permits bone anchors to be implanted into bone with reduced occurrence of contamination and, thus, decreases the likelihood of bone infection and associated biological complications.

The protective cover of the present invention can be advantageously made of a bioabsorbable material. A suitable bioabsorbable material erodes or is dissolved within the patient after a predetermined period of time. Thus, following implantation of the bone anchor, the protective cover material is absorbed by natural biological processes. Use of bioabsorbable materials reduces the risk of introducing foreign particles in the patient's body and undesirable immunogenic response, commonly associated with the use of non-biodegradable materials, such as rubber, latex or silicone.

Additionally, the protective cover of the present invention is generally ellipsoidal and its diameter only slightly exceeds the length and/or the width of the bone anchor. Because of its convenient size and low profile, the protective cover of the present invention performs the above-described functions without compromising maneuverability and ease of use.

In one aspect, the invention features a protective cover for encapsulating a bone anchor comprising a generally ellipsoidal mass. In some embodiments the generally ellipsoidal mass is deformable. In other embodiments, the generally ellipsoidal mass is substantially brittle. In some embodiments the generally ellipsoidal mass comprises a bioabsorbable material.

Nonlimiting examples of useful polymers include the following: polyglycolic acid (PGA), polyactic acid (PLA), poly (dioxanone) (PDO), poly (dl-lactide) (LPLA), poly (dl-lactide) (DLPLA), poly (glycolide-co-trimethylene carbonate) (PGA-TMC), poly (1-lactide-co-glycolide) (PGA-LPLA), poly (dl-lactide-co-glycolide) (PGA-DLPLA), poly (1-lactide-co-dl-lactide) (LPLA-DLPLA), poly(glycolide-co-trimethylene carbonate-co-dioxanone) (PDO-PGA-TMC), poly(ε-caprolactone), poly (lactide-co-glycolide), poly(SA-HDA anhydride), poly(orthoester), and polyglyconate.

In some embodiments of the invention, the generally ellipsoidal mass also contains an antibiotic or a combination of antibiotics, which may be disposed within the bioabsorbable material or may be applied to at least one surface of the protective cover. Nonlimiting examples of useful antibiotics include the following: nafcillin, aminoglycoside, ciprofloxin, piperacillin/tazobactam, ampicillin/sulbactum, vancomycin, cephalosporin, TMP/SMX, ampicillin, genitamicin, tobramycin, and ciprofloxacin

In another aspect, the present invention features a method of inserting a bone anchor into a bone, including the steps of providing the bone anchor encapsulated by a protective cover; locating the bone anchor implantation site on the bone; and applying a force to the bone anchor to cause the bone anchor to penetrate the protective cover and implant the bone anchor into the bone.

In yet another aspect, the present invention features a medical device for attaching soft tissue to a bone, which consists of a bone anchor and a solid mass formed from a biocompatible material, wherein said bone anchor is encapsulated in said mass, which forms a protective cover.

The foregoing and other objects, aspects, features, and advantages of the invention will become more apparent from the following detailed description and the appended claims.

### DEFINITIONS

In order to more clearly and concisely point out the subject matter of the claimed invention, the following definitions are provided for specific terms used in the following written description and appended claims.

Deformable material. As used herein, a “substantially deformable material” means a solid material capable of a change in shape without breaking.

Brittle material. As used herein, a “brittle material” means a solid material, which breaks when subjected to a stress without an appreciable prior deformation. Such a brittle material is expected lose structural integrity and break upon the application of less than 5 pounds of force on the material.

Biocompatible material. As used herein, it material is “biocompatible” when it is compatible with living tissue by virtue of a substantial lack of toxicity or relative inability to cause immunological rejection.

Bioabsorbable material. As used herein, a material is “bioabsorbable” when, in addition to being biocompatible, it is capable of disintegration in the human body by hydrolysis, enzymatic cleavage or other natural processes. As used herein, the term ““bioabsorbable material” includes any biodegradable material.

Antimicrobial material. As used herein, an antimicrobial material” is one which destroys or inhibits the growth of microscopic and/or submicroscopic organisms.

Push-in bone anchor. As used herein, a “push-in bone anchor” is a bone anchor, which is implanted into the bone by a device, which imparts a linear force upon the bone anchor. In general, there are two types of push-in bone anchors: the staple type, which is made of shape memory material, which is typically driven through bone and then adopts a curved configuration, which fixes the anchor to the bone. A second type of push-in bone anchor typically includes a piercing leading edge (e.g., a trocar tip) to penetrate the bone tissue and a crown or wings to resist pull out.

Screw-in bone anchor. As used herein, a “screw-in bone anchor” is a threaded bone anchor, which is implanted into the bone by a device, which imparts a rotational force upon the bone anchor. Screw type anchors are typically driven into but not through the outer cortex of the bone. The screw-in anchor is held in place by the threads of the screw.

### DETAILED DESCRIPTION OF THE INVENTION

The protective cover of the present invention is adapted to reduce injuries resulting from bone anchor implantation procedures, for example, puncture of a doctor's glove, abrasion of the patient's tissue or insertion of the bone anchor tip into tissue other than the target tissue. The device of the present invention may further decrease the risk of infection by encapsulating the bone anchor in a solid mass into which one or more antibiotics have been incorporated. To ensure a sterile barrier around the bone anchor and to shield the bone anchor from microorganisms in the surgical field surrounding the implantation site, it is preferred that the protective cover completely encapsulates the bone anchor.

The bone anchor cover of the present invention consists of a solid mass of biocompatible material, which substantially encapsulates the bone anchor, covering the sharp edges of the bone anchor. In a preferred embodiment, however, the cover does not encapsulate the shaft of the bone anchor so that the anchor with the protective cover may be releasably attached to the mount of a bone anchor implantation device. Additionally, the diameter of the protective cover of the present invention only slightly exceeds the length and width of the bone anchor. Because of its low profile, the protective cover of the present invention does not compromise maneuverability and ease of use of the bone anchor.

In one embodiment of the present invention, the bone anchor consists of a generally ellipsoidal solid mass, which is substantially deformable. In such an embodiment the protective cover deforms during implantation and partially fills the opening formed by the bone anchor, and the deformed protective cover continues to cover the implantation site providing a sterile barrier until the patient's body absorbs the bone anchor cover. The deformable mass embodiment of the present invention is preferably used in conjunction with screw-in bone anchors. In use, such a screw-in type bone anchor encapsulated in a deformable protective cover is driven into the bone, the leading tip punctures the cover, but the cover remains associated with the anchor and fills in the recesses created by the threads. The deformable cover remains in place around the anchor until absorbed by the body and/or the bone tissue grows into the recesses created by the screw threads.

In another series of embodiments the protective cover is substantially brittle. The brittle mass embodiment of the present invention is preferably used in conjunction with push-in bone anchors. In use, such a push-in type bone anchor encapsulated in a brittle protective cover is driven into the bone, the trocar tip of the anchor punctures the cover and the bone, and, consequently, the protective cover breaks into pieces. After the implantation, the pieces of the protective cover remain in the body until they disintegrate and are absorbed.

In preferred embodiments of the present invention, the biocompatible material used to form the protective cover is also bioabsorbable. A suitable bioabsorbable material dissolves within the patient after a predetermined period of time. In a preferred embodiment, the bioabsorbable material dissolves within 90 days. In a more preferred embodiment, the bioabsorbable material dissolves within 60 days. In a most preferred embodiment, the bioabsorbable material dissolves within 30 days. Use of bioabsorbable materials reduces the risk of introducing foreign particles in the patient's body, which could trigger undesirable immunogenic response, commonly associated with the use of non-biodegradable materials, such as rubber, latex or silicone.

Preferred bioabsorbable materials include polymeric materials such as polyesters, polyorthoesters, or polyanhydrides, including polymers or copolymers of glycolic acid, lactic acid, or sebacic acid. More generally, preferred materials include polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy acids (e.g., (COOH)(CH2)n(OH) or (COOH)(CRiRj)n(OH), where n is an integer between about 1 and 20, and each Ri and Rj is independently selected from the group consisting of —H, —OH, —SH, —NH2, the halogens, the side chains of the naturally occurring amino acids, and any straight chain or branched, substituted or unsubstituted, saturated or unsaturated, low molecular weight (e.g., C1-C14) alkanyl, haloalkyl, thioalkyl, aminoalkyl, aryl, aralkyl, alkenyl, aralkenyl, heteroaryl, or alkoxy group, or a secondary or tertiary amine substituted with such groups) or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids (e.g., (COOH)(CH2)n(COOH) or (COOH)(CRiRj)n(COOH), where n is an integer between about 1 and 20, and each Ri and Rj is independently selected from the group consisting of —H, —OH, —SH, —NH2, the halogens, the side chains of the naturally occurring amino acids, and any straight chain or branched, substituted or unsubstituted, saturated or unsaturated, low molecular weight (e.g., C1-C14) alkanyl, haloalkyl, thioalkyl, aminoalkyl, aryl, aralkyl, alkenyl, aralkenyl, heteroaryl, or alkoxy group, or a secondary or tertiary amine substituted with such groups). Polymers including mixtures of ester and anhydride bonds (e.g., copolymers of glycolic and sebacic acid) may also be employed.

Other biocompatible bioabsorbable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials.

The currently preferred bioabsorbable materials are polyglycolic acid (PGA), polyactic acid (PLA), poly (dioxanone) (PDO), poly (l-lactide) (LPLA), poly (di-lactide) (DLPLA), poly (glycolide-co-trimethylene carbonate) (PGA-TMC), poly (l-lactide-co-glycolide) (PGA-LPLA), poly (dl-lactide-co-glycolide) (PGA-DLPLA), poly (l-lactide-co-dl-lactide) (LPLA-DLPLA), poly(glycolide-co-trimethylene carbonate-co-dioxanone) (PDO-PGA-TMC), poly(ε-caprolactone), poly(lactide-co-glycolide), poly(SA-HDA anhydride), poly(orthoester), and polyglyconate.

The protective cover of the present invention also provides a sterile antimicrobial barrier around the bone anchor and shields the bone anchor from contacting microorganisms in the surgical field surrounding the implantation site prior to and during insertion. The cover additionally provides prolonged supplemental antimicrobial protection of the implantation site after the bone anchor is inserted. In some embodiments, the protective cover includes an antibiotic or a blend of antibiotics that have complementary activity to reduce the occurrence of contamination and, therefore, decrease the likelihood of bone infection and associated biological complications.

In some embodiments, an antibiotic may be topically applied to a part of the surface of the protective cover, which will come into contact with the bone during implantation. In an alternative embodiment, one or more antibiotics maybe disposed within the bioabsorbable material to form the protective cover. Such a combination of an antibiotic and a bioabsorbable material ensures the release of antibiotic in the patient's body over a period of time. This release of antibiotic occurs as the patient's body absorbs the bioabsorbable material, which forms the protective cover of the present invention. This “time-release” effect effectively prolongs the duration of beneficial application of the antibiotic to the implantation site. The rate of such release depends upon the degradation rate of the bioabsorbable material and upon the concentration of antibiotic in the bioabsorable material. In some embodiments the bioabsorbable material is absorbed with a half-life of between 3-22 days. In preferred embodiments the bioabsorbable material is absorbed with a half-life of between 5-14 days. In a more preferred embodiment the bioabsorbable material is absorbed with a half-life of between 5-8 days. In a most preferred embodiment the bioabsorbable material is absorbed with a half-life of 7 days.

Furthermore, to ensure sterility of the protective covers, it is advantageous to prepare protective covers of various sizes and dispense them to physicians in individual sterile packages. A physician can thus choose the cover according to the size of the bone anchor to be implanted, open the package and place the protective cover on the bone anchor shortly before the surgical procedure.

In some embodiments, a bone anchor with or without an associated suture may be sterilized, encapsulated in a protective cover, and packaged in the sterile wrapping, using methods known in the art. Subsequently, the bone anchor encapsulated in the protective cover may be provided to the physician as a single article of manufacturing, thus, eliminating the step of applying the protective cover to the bone anchor. Such an embodiment ensures the proper match between the size of the bone anchor and the size of the protective cover. Furthermore, elimination of the step of manual application of the protective cover to the bone anchor reduces the time necessary for preparation for the implantation procedure and reduces the risk of the injury to a physician or a nurse assembling the bone anchor implantation device. In addition, bone anchors encapsulated in a protective cover have substantially the entire surface area of the anchor (excluding the shaft) in contact with the bioabsorbable material combined with an antibiotic for a period of time prior to the implantation procedure, which further ensures the to sterility of the implantation procedure.

The protective cover of the present invention can be advantageously utilized with a bone anchor implantation device in a method of inserting the bone anchor using such device. In the preferred embodiments of the present invention, a bone anchor is releasably attached to a bone anchor implantation device. The protective cover made of a deformable bioabsorbable material encapsulates the bone anchor so that the sharp edges of the bone anchor are covered, but leaving the shaft of the anchor exposed so that the anchor can be mounted on the bone anchor implantation device. In some preferred embodiments, an antibiotic is disposed within the bioabsorbable material of the protective cover. In alternative preferred embodiments, once a physician locates a bone anchor implantation site on the bone, a force is applied to the bone anchor implantation device causing the bone anchor to penetrate the protective cover and implant the bone anchor into a bone. In such embodiments using a deformable protective cover, the deformed protective cover fills the opening formed by the bone anchor, providing a sterile antimicrobial barrier around the bone anchor and prolonged supplemental antimicrobial protection of the implantation site. In a preferred embodiment of the present invention, an absorption rate of a bioabsorbable material is comparable to a bone growth rate. In a more preferred embodiment of the present invention, an absorption rate of a bioabsorbable material is less than a bone growth rate. This ensures that the bone tissue and the bone anchor remain in contact with the bioabsorbable material combined with an antibiotic until the bone tissue grows into the recesses created by the bone anchor, and prolongs the supplemental antimicrobial protection of the implantation site.

In other embodiment of the present invention the protective cover is made of a brittle bioabsorbable material, in such embodiments, a push-in type bone anchor is encapsulated within a brittle protective cover, when the encapsulated bone anchor is driven into the bone, the tip of the anchor punctures the cover and the bone and, consequently, the protective cover breaks into pieces. After the implantation, the pieces of the protective cover remain in the body until they are absorbed.

Variations, modifications, and other implementations of what is described herein will occur to those of ordinary skill in the art without departing from the spirit and the scope of the invention as claimed. Accordingly, the invention is to be defined not by the preceding illustrative description but instead by the spirit and scope of the following claims.

What is claimed is:

1. A medical device for attaching soft tissue to a bone comprising a bone anchor and a protective cover formed from a solid mass of biocompatible material, wherein said bone anchor is substantially encapsulated in said mass.

2. The protective cover of claim 1, wherein said mass is substantially deformable.

3. The protective cover of claim 1, wherein said mass is substantially brittle.

4. The protective cover of claim 1, wherein said mass comprises a bioabsorbable material.

5. The protective cover of claim 4, wherein said bioabsorbable material is selected from the group consisting of cross-linked alginated gel, cross-linked collagen, cross-linked hyaluronic acid hydrogel, polylactic-co-glycolic acid, polylactic acid, polyglycolic acid, polyurethane.

6. The protective cover of claims 1, further comprising an antimicrobial material.

7. The protective cover of claim 6, wherein said antimicrobial material comprises an antibiotic.

8. The protective cover of claim 7, wherein said antibiotic is selected from the group consisting of nafcillin, aminoglycoside, ciprofloxin, piperacillin/tazobactum, ampicillin/sulbactum, vancomycin, cephalosporin, TMP/SMX, ampicillin, gentamicin, tobramycin, and ciprofloxacin.

9. The protective cover of claim 8, wherein said antibiotic is disposed within said bioabsorbable material to form said cover.

10. The protective cover of claim 8, wherein said antibiotic is applied to at least one surface of said protective cover.

11. A method of inserting a bone anchor into a bone, comprising:
(a) providing a bone anchor;
(b) providing a protective cover adapted to encapsulate said bone anchor,
(c) encapsulating said bone anchor in said protective cover;
(d) locating a bone anchor implantation site on a bone; and
(e) causing said bone anchor to penetrate said protective cover and implant in said bone.

12. The method of claim 11, wherein said bone anchor is encapsulated in said protective cover prior to engagement of said bone anchor to an implantation device.

13. The method of claim 11, wherein said bone anchor is encapsulated in said protective cover after engagement of said bone anchor to an implantation device.

14. The method of claim 11, wherein said protective cover for encapsulating a bone anchor comprises a generally ellipsoidal mass.

15. The method of claim 14, wherein said mass is substantially deformable.

16. The method of claim 14, wherein said mass is substantially brittle.

17. The method of claim 14, wherein said mass comprises a bioabsorbable material.

18. The method of claim 17, wherein said bioabsorbable material is selected from the group consisting of cross-linked alginated gel, cross-linked collagen, cross-linked hyaluronic acid hydrogel, polylactic-co-glycolic acid, polylactic acid, polyglycolic acid, polyurethane.

19. The method of claims 18, wherein said protective cover further comprises an antibiotic.

20. The method of claim 19, wherein said antibiotic is selected from the group consisting of nafcillin, aminoglycoside, ciprofloxin, piperacillin/tazobactum, ampicillin/sulbactum, vancomycin, cephalosporin, TMP/SMX, ampicillin, gentamicin, tobramycin, and ciprofloxacin.

21. The method of claim 20, wherein said antibiotic is disposed within said bioabsorbable material to form said cover.

22. The method of claim 20, wherein said antibiotic is applied to at least one surface of said protective cover.
